(19) United States
(12) Patent Application Publication
(10) Pub. No.: US 2011/0070579 A1
(43) Pub. Date: Mar. 24, 2011

(54) POLYKETIDE SYNTHASE-NONRIBOSOMAL PEPTIDE SYNTHETASE GENE

(76) Inventors: Masafumi Tokuoka, Higashi hiroshima-shi (JP); Tadashi Takahashi, Noda-shi (JP); Yasuji Koyama, Noda-shi (JP); Isao Fujii, Shiwa-gun (JP); Yasuyo Seshime, Kawagoe-shi (JP)

(21) Appl. No.: 12/672,243

(22) PCT Filed: Oct. 16, 2008
(86) PCT No.: PCT/JP2008/068698

§ 371 Date: Feb. 4, 2010

(30) Foreign Application Priority Data
Oct. 19, 2007 (JP) 2007-272465

(51) Int. Cl. C12Q 1/68; C12N 15/52; C12N 9/00; C12N 15/74; C12N 1/15

(52) U.S. Cl. 435/6; 536/23.2; 435/183; 435/471; 435/256.1; 435/256.3

(57) ABSTRACT

The objective is to provide a method for rapidly, and highly accurately performing genetic detection of the cyclopiazonic acid-producing ability in a strain belonging to genus Aspergillus, etc. as well as a transformant (strain), etc. that does not produce cyclopiazonic acid or a precursor thereof, cycloacetoacetyl L-tryptophan (CAT). A polynucleotide encodes the following polypeptides: (1) a polypeptide comprising the amino acid sequence depicted in SEQ ID NO: 2; (2) a polypeptide comprising an amino acid sequence with one or several amino acids deleted, substituted or added in the amino acid sequence depicted in SEQ ID NO: 2 and having a polyketide synthase-nonribosomal peptide synthetase activity; or (3) a polypeptide having 90% or more homology (identity) as an overall average with the amino acid sequence depicted in SEQ ID NO: 2 and having a polyketide synthase-nonribosomal peptide synthetase activity.

### FIELD OF THE INVENTION

The present invention relates to a novel polyketide synthase-nonribosomal peptide synthetase gene, a method for producing by gene recombination a bacterial transformant that does not produce cyclopiazonic acid, and a method for detecting the cyclopiazonic acid-producing ability of Aspergillus bacteria or Penicillium bacteria by detecting the presence of a base sequence included in said gene's 3′ region and its 3′ downstream region.

### BACKGROUND OF THE INVENTION

Rice molt fungi (Aspergillus) such as Aspergillus sojae (Aspergillus sojae) and Aspergillus oryzae (Aspergillus oryzae) are filamentous bacteria that have been industrially widely used for the brewing of traditional foods such as soy sauce, sake, and bean paste and production of enzymes, etc. and that, due to the recent determination of full-genome sequences of rice molt fungi (Aspergillus oryzae) and the development of exhaustive gene expression analysis, etc. using microarrays, is expected to have the effect of improving the productivity and proliferation rate of enzymes, etc. by genetic engineering modification, particularly modification at the chromosomal level.

As mentioned above, in addition to Aspergillus being utilized as a host microorganism for protein production such as food manufacturing and manufacturing of enzymes, its secondary metabolic substances are utilized as pharmacological products and precursors thereof. However, such secondary metabolic substances include mycotic toxins known as mycotoxins.

Cyclopiazonic acid, which is one type of such mycotoxins, is a neurological toxin inhibiting ATP-dependent calcium transporters that was first found in Penicillium cyclopium. Its toxicity is known to be LD50=36 mg/kg (oral administration in rats) and it is believed that it is one of the causative toxins in the turkey X disease in England in 1960 in which over 100,000 turkeys died from mycotic toxins. As fungi that produce cyclopiazonic acid, in addition to above Penicillium cyclopium, some bacterial strains are known, such as Penicillium camenberti, Aspergillus flavus, Aspergillus oryzae, and Aspergillus tamarii.

It is known that in Aspergillus oryzae, used for manufacturing soy sauce, bean paste, and sake, etc. and Penicillium camenberti, used for manufacturing cheese, some strains thereof produce cyclopiazonic acid. Thus, in order to ensure the safety of brewed foods, it is necessary to discriminate cyclopiazonic acid-producing bacteria and eliminate them. At the same time, these traditional brewed/fermented foods are produced on various scales at present, and the actual situation is that manufacturers with different technical capabilities and facilities select strains to be used at their individual discretion. Thus, a method for discriminating cyclopiazonic acid-producing bacteria that is as simple and accurate as possible is desired.

The easiest method of discrimination that is conventionally known is a method for discriminating by culturing a test strain in an agar medium, wherein colonies become red with the presence of cyclopiazonic acid (Patent Document 1), but in this method, it is the productivity under the culturing conditions of an agar medium that is determined, and therefore it is unknown whether cyclopiazonic acid is produced during the actual brewing process. At the same time, as a more accurate method, it has been reported that the method in which, in genus Penicillium and genus Claviceps, cyclopiazonic acid-producing bacteria are discriminated by detecting a gene of 4-dimethylallyltriptophan synthase, which is one of the enzymes that catalyze cyclopiazonic acid biosynthesis, using a polymerase chain reaction (hereinafter also referred to as “PCR”) is effective (Non-Patent Document 1). However, regarding genus Aspergillus, our research clarified that there is a strain that retains dimethylallyl cycloacetoacetyl-L-tryptophan synthase, which is a homologous gene of this gene, in non-producing bacteria also, and therefore a similar method cannot always be utilized in genus Aspergillus.

Moreover, a method for developing a mycotoxin non-producing strain by mutation and destruction of a gene using a gene manipulation technique, etc. in order to obtain a substance producing host that does not produce mycotoxins, such as cyclopiazonic acid, has been reported (Patent Documents 2 and 3). However, in the evaluation of a strain created in these methods, only molecules, which are the final products, are focused upon, and therefore even a strain in which a precursor thereof is synthesized may be recognized as a mycotoxin non-producing strain. However, for example, sterigmatocystin, which is a precursor of aflatoxin, a type of mycotoxin, is a strong toxic substance, although it is weaker than aflatoxin Therefore, as a fungus used for substance production and food manufacturing, it is expected to enhance safety by using not only mycotoxins, which are the final product, but also precursors thereof, and preferably a strain inactivated during synthesis at an earlier stage, losing its mycotoxin productivity.

Previously, because rice molt fungi have low homologous recombination frequency, in conventional methods, it has been very difficult to create a large region deleting strain for any region of a chromosome. For example, even when one vector is incorporated into any region on a chromosome, because the frequency of homologous recombination is low, which is 1 to 2%, it is necessary to obtain some dozens to a hundred strains of transformants and among them, obtain a target strain. The present inventors clarified in their recent research that destroying a gene involved in non-homologous recombination improves the gene targeting frequency (homologous recombination frequency) to a large extent (Patent Document 4).
Patent Document 1: Japanese Patent Application Publication No. Hei 05-030994
Patent Document 2: WO2000/039322
Patent Document 3: Japanese Patent application Publication No. 2002-533133
Patent Document 4: Japanese Patent Application Publication No. 2007-222055
Non-Patent Document 1: Use of polymerase chain reaction for searching for producers of ergot alkaloids from among microscopic fungi, Boichenko L V, Boichenko D M, Vinokurova N G, Reshetilova T A, Arinbasarov M U. Mikrobiologiia. 2001 May-June; 70(3):360-4.

### SUMMARY OF THE INVENTION

### Problem to be Resolved by the Invention

A major objective of the present invention is to provide a method for rapidly and highly accurately detecting a strain genetically having cyclopiazonic acid-producing ability in Aspergillus bacteria or Penicillium bacteria. Moreover, the present invention provides a primer for PCR that allows for accurate and easy detection of cyclopiazonic acid-producing bacteria. A further major objective of the present invention is to provide, in Aspergillus bacteria or Penicillium bacteria, a transformant (strain) that does not produce cyclopiazonic acid or cycloacetoacetyl-L-tryptophan (CAT), which is a precursor thereof, by producing transformed bacteria in which the genes of an enzyme that catalyzes the first reaction process of the cyclopiazonic acid biosynthesis pathway are destroyed.

### Means of Solving the Problem

The present inventors conducted a study in order to solve the above problems and consequently identified a polyketide synthase-nonribosomal peptide synthetase gene, which is an enzyme that catalyzes the first stage of the cyclopiazonic acid biosynthesis pathway, clarified the role of said gene in cyclopiazonic acid biosynthesis by comparing said gene and the base sequence of its 3′ downstream region between a cyclopiazonic acid-producing strain and a cyclopiazonic acid non-producing strain, and completed the present invention.

In other words, the present invention relates to each of the following aspects.
[Aspect 1] A polynucleotide encoding the following polypeptides:
(1) a polypeptide comprising the amino acid sequence depicted in SEQ ID NO: 2;
(2) a polypeptide comprising an amino acid sequence with one or several amino acids deleted, substituted or added in the amino acid sequence depicted in SEQ ID NO: 2 and having a polyketide synthase-nonribosomal peptide synthetase activity; or
(3) a polypeptide having 90% or more homology (identity) as an overall average with the amino acid sequence depicted in SEQ ID NO: 2 and having a polyketide synthase-nonribosomal peptide synthetase activity.
[Aspect 2] A polynucleotide comprising the following polynucleotides:
(1) polynucleotides comprising the base sequence depicted in SEQ ID NO: 1;
(2) polynucleotides hybridizing under stringent conditions with polynucleotides comprising a base sequence complementary to the base sequence depicted in SEQ ID NO: 1 and encoding a polypeptide having a polyketide synthase-nonribosomal peptide synthetase activity.
[Aspect 3] A polynucleotide according to Aspect 1 or 2 that is genomic DNA included in the genome of Aspergillus oryzae.
[Aspect 4] A polynucleotide according to Aspect 1 or 2 that is cDNA.
[Aspect 5] Polyketide synthase-nonribosomal peptide synthetase comprising the following polypeptides:
(1) a polypeptide comprising the amino acid sequence depicted in SEQ ID NO: 2;
(2) a polypeptide comprising an amino acid sequence with one or several of the amino acids deleted, substituted or added in the amino acid sequence depicted in SEQ ID NO: 2 and having a polyketide synthase-nonribosomal peptide synthetase activity; or
(3) a polypeptide having 90% or more homology (identity) as an overall average with the amino acid depicted in SEQ ID NO: 2 and having a polyketide synthase-nonribosomal peptide synthetase activity.
[Aspect 6] A method for producing by gene manipulation a transformant of a microorganism belonging to genus Aspergillus or genus Penicillium that does not produce cyclopiazonic acid.
[Aspect 7] The method according to Aspect 6, wherein the transformant that does not produce cyclopiazonic acid is a bacterium that does not produce cycloacetoacetyl L-tryptophan.
[Aspect 8] The method according to Aspect 6 or 7, wherein the transformant that does not produce cyclopiazonic acid is a bacterium that does not express polyketide synthase-nonribosomal peptide synthetase.
[Aspect 9] The method according to any one of Aspects 6 to 8, wherein the gene manipulation destroys the polynucleotide encoding the polyketide synthase-nonribosomal peptide synthetase according to Aspect 1 or 2.
[Aspect 10] The method according to Aspect 9, wherein the gene manipulation is performed on a strain in which a Ku gene involved in non-homologous recombination is destroyed.
[Aspect 11] The method according to any one of Aspects 6 to 10, wherein the microorganism is an Aspergillus oryzae strain.
[Aspect 12] The method according to Aspect 11, wherein an Aspergillus oryzae strain in which the homologous recombination frequency has been elevated is used.
[Aspect 13] The method according to Aspect 12, wherein a transformed bacterium in which the homologous recombination frequency has been elevated is an Aspergillus oryzae A4177K strain.
[Aspect 14] A transformant that does not produce cyclopiazonic acid, obtained by the method of manufacturing according to any one of the Aspects 6 to 13.
[Aspect 15] The transformant that does not produce cyclopiazonic acid according to Aspect 14 that is an Aspergillus oryzae strain.
[Aspect 16] A method for discriminating cyclopiazonic acid-producing ability, wherein a partial base sequence of a polynucleotide included in the 3′ region of the polynucleotide encoding the polyketide synthase-nonribosomal peptide synthetase according to Aspect 1 or 2 or in the region from the stop codon to the telomere of said polynucleotide is detected, and cyclopiazonic acid-producing ability in an Aspergillus strain or a Penicillium strain is discriminated based on the presence or absence of said partial base sequence.
[Aspect 17] A method for identifying a strain that does not produce cyclopiazonic acid, wherein a partial base sequence of a polynucleotide included in the region on the 3′ side of the polynucleotide encoding the polyketide synthase-nonribosomal peptide synthetase according to Aspect 1 or 2 is detected, and a bacterium that does not produce cyclopiazonic acid in an Aspergillus strain or a Penicillium strain is identified based on the presence or absence of said partial base sequence.
[Aspect 18] The method according to Aspect 16 or 17, wherein the Aspergillus strain is Aspergillus oryzae.
[Aspect 19] The method according to any one of Aspects 16 to 18, wherein the 3′ region of the polynucleotide encoding the polyketide synthase-nonribosomal peptide synthetase is the 4,217th to the 11,721st in the base sequence depicted in SEQ ID NO: 1.
[Aspect 20] The method according to Aspect 16, wherein the polynucleotide region included between the polynucleotide encoding the polyketide synthase-nonribosomal peptide synthetase and the telomere sequence present in its 3′ downstream region has the base sequence depicted in SEQ ID NO: 3.
[Aspect 21] The method according to any one of Aspects 16 to 20, wherein the presence or absence of the partial base sequence of the polynucleotide is detected by a PCR.
[Aspect 22] The method according to any one of Aspects 16 to 20, wherein the presence or absence of the partial base sequence of the polynucleotide is detected by a Southern analysis.

### Effects of the Invention

In Aspergillus bacteria, a polyketide synthase-nonribosomal peptide synthetase (PKS-NRPS) gene, which is an enzyme that catalyzes the first stage of a cyclopiazonic acid biosynthesis pathway, was first identified by the present invention. In addition, in bacteria that did not produce cyclopiazonic acid, it was found that the 3′ end side of the 4,217th base and thereafter of said PKS-NRPS gene is deleted, the stop codon is not included, and a telomere repeat sequence is added to the 3′ side of said base.

In consequence, in a strain targeted for determination, it is possible to detect a partial base sequence of a polynucleotide included in the 3′ region of a polynucleotide encoding PKS-NRPS of the present invention or the region from the stop codon to the telomere of said polynucleotide, to discriminate cyclopiazonic acid-producing ability in an Aspergillus strain or a Penicillium strain based on the presence or absence of said partial base sequence, and to identify a bacterium that does not produce cyclopiazonic acid.

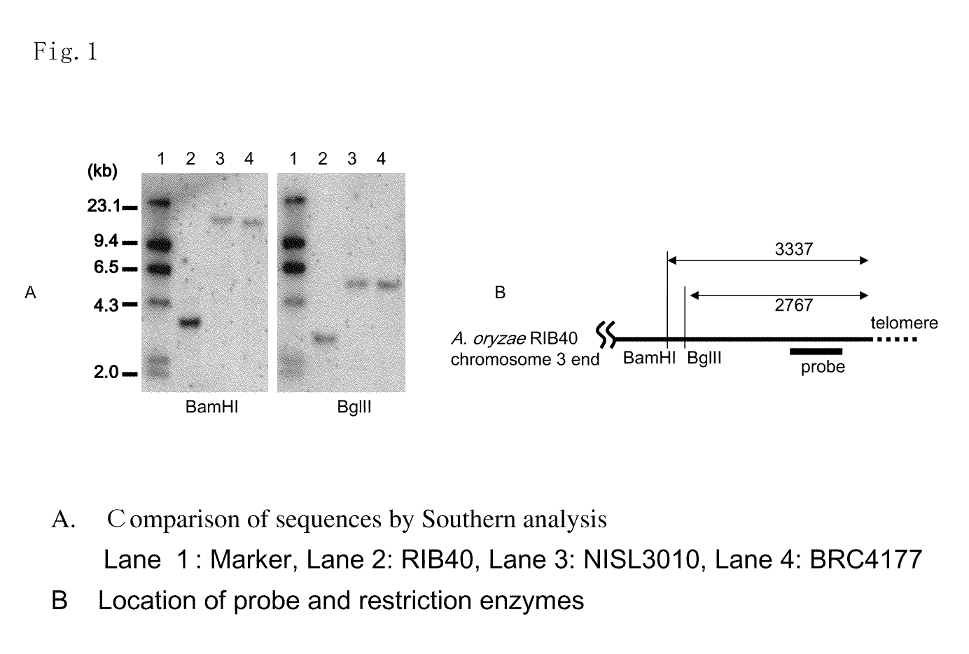

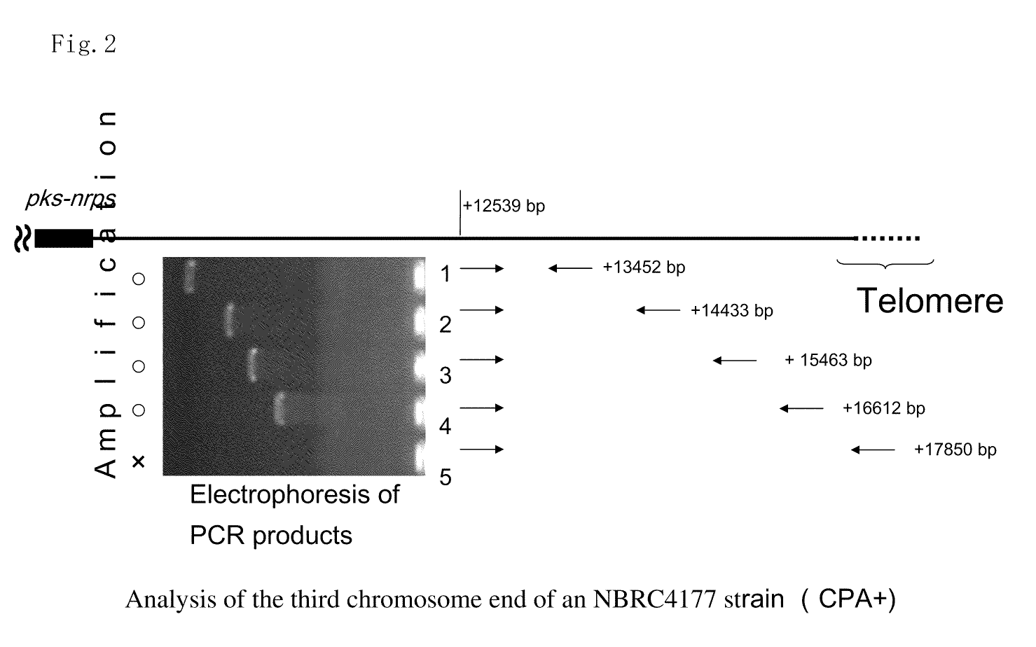

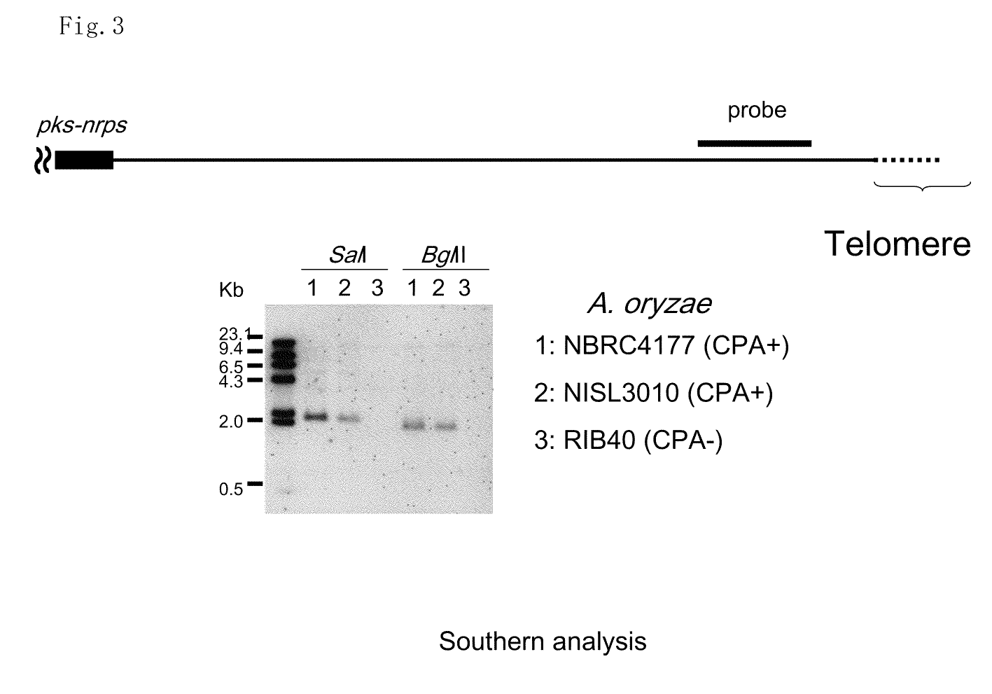

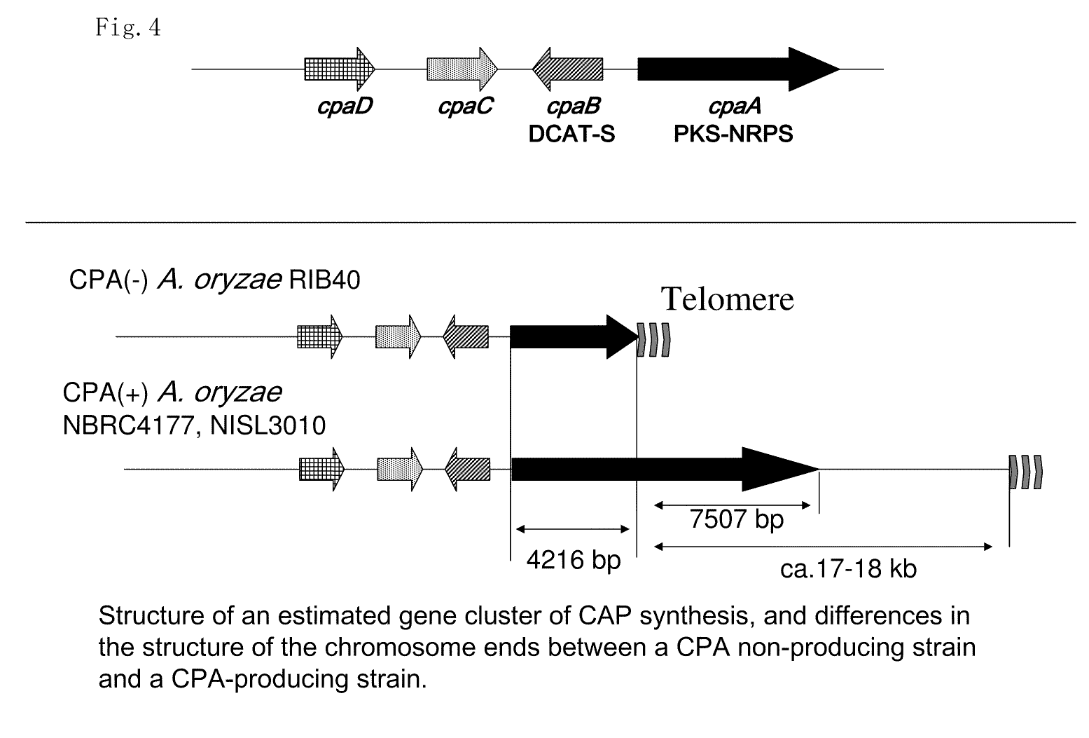

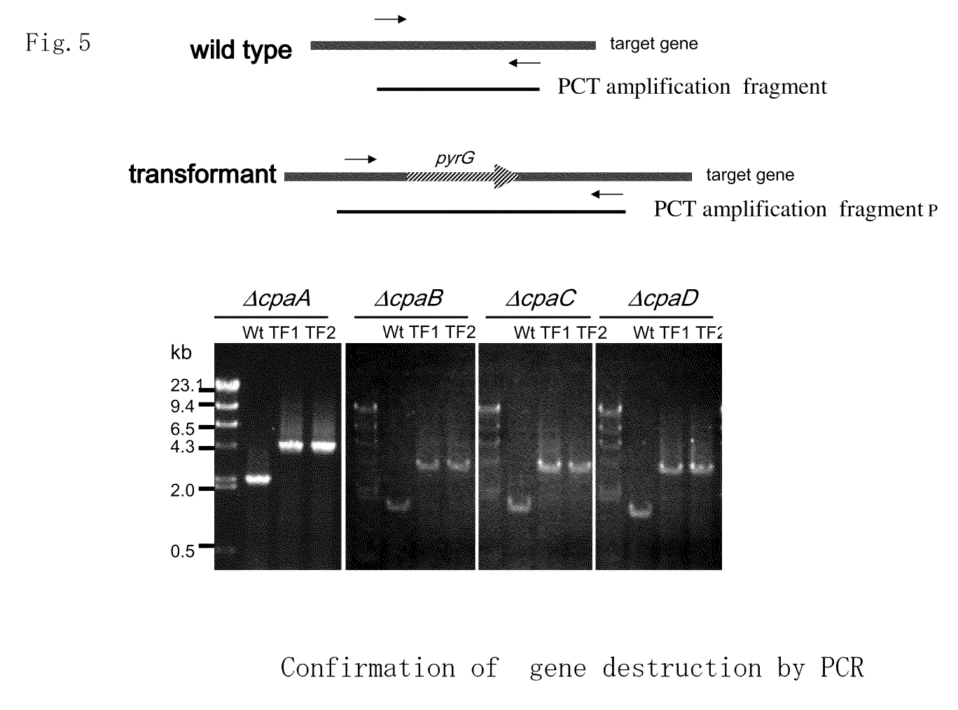

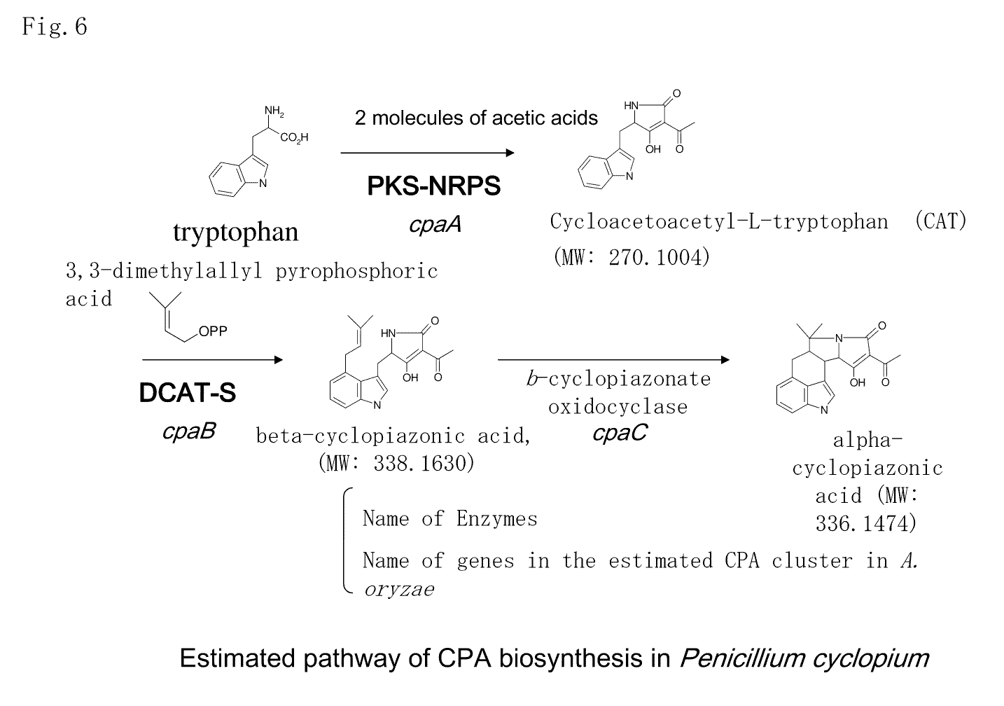

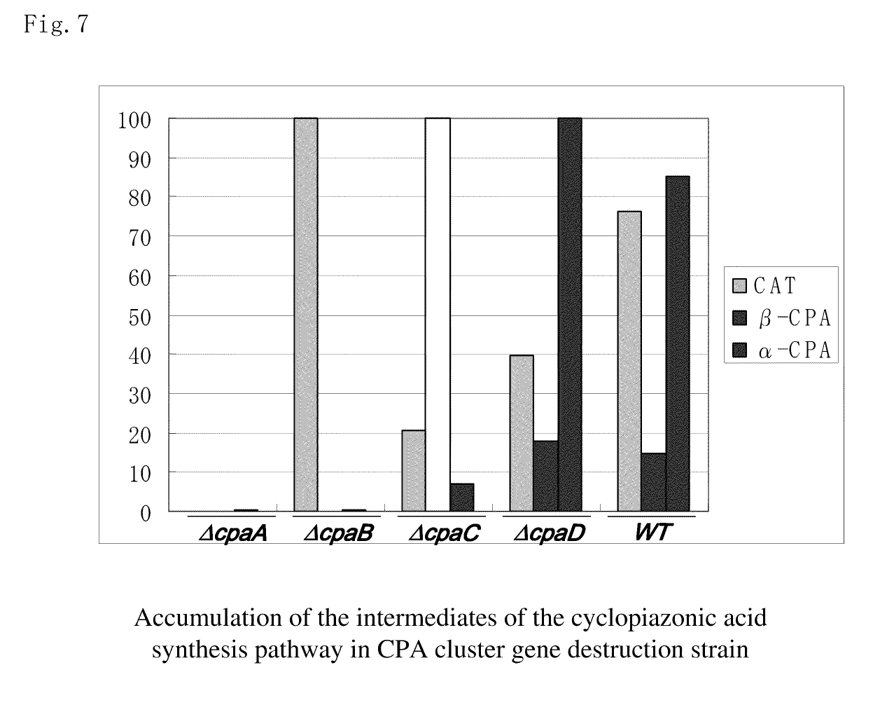

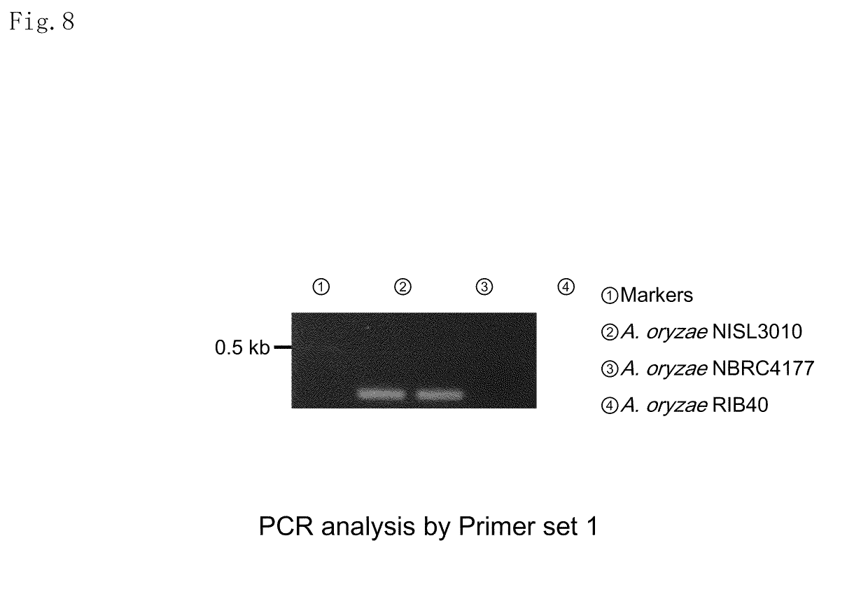

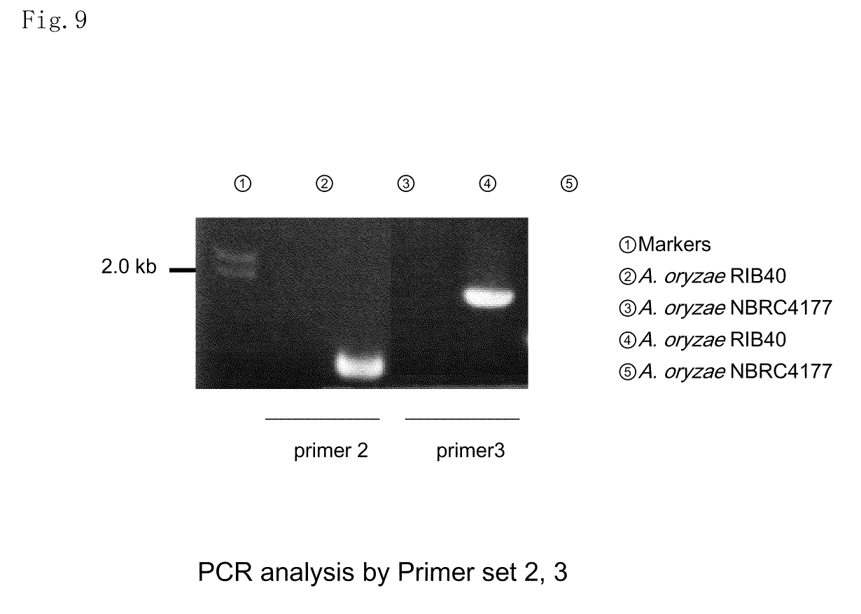

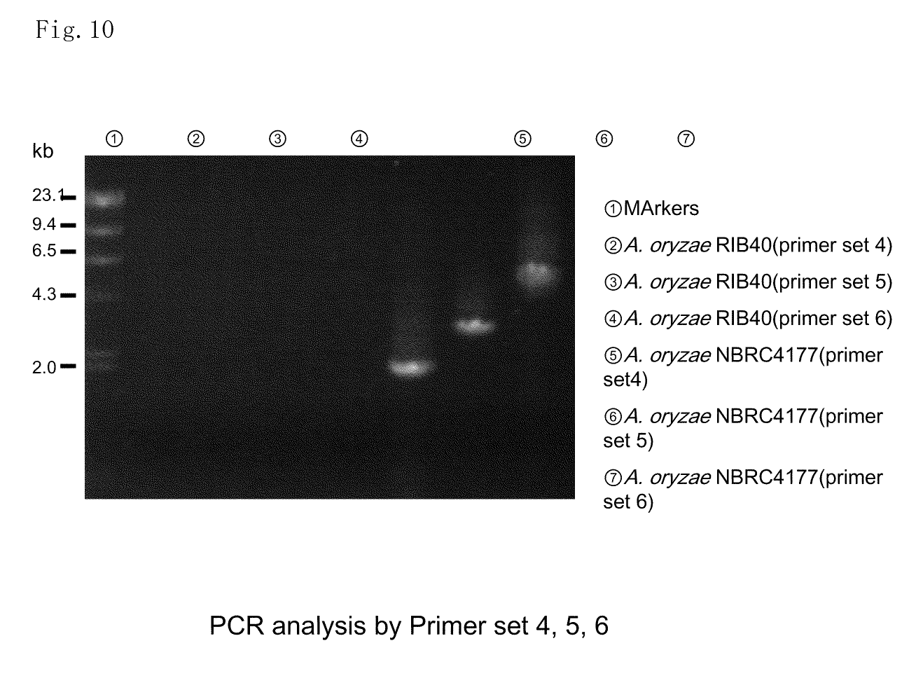

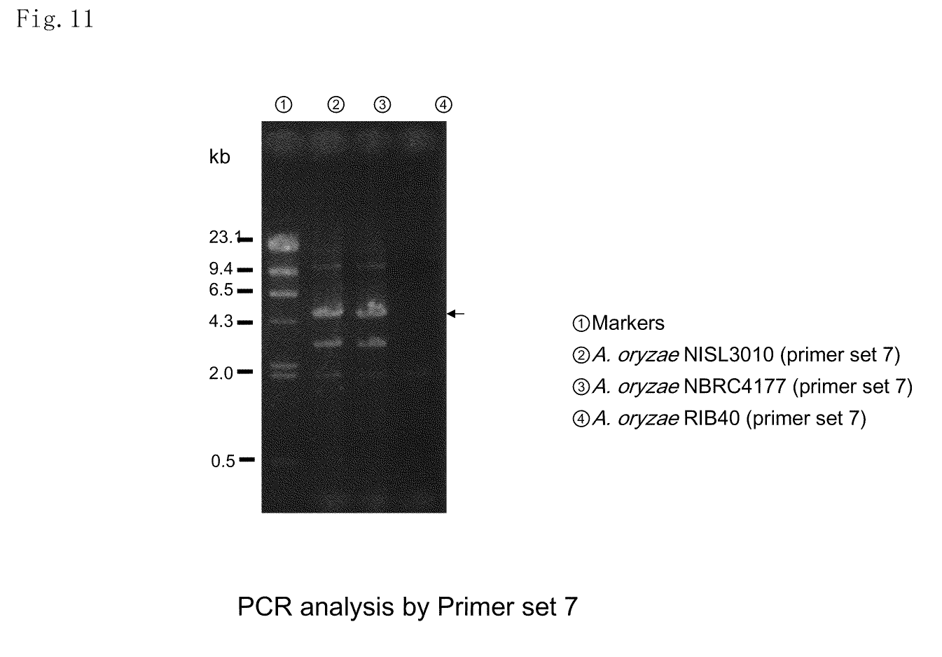

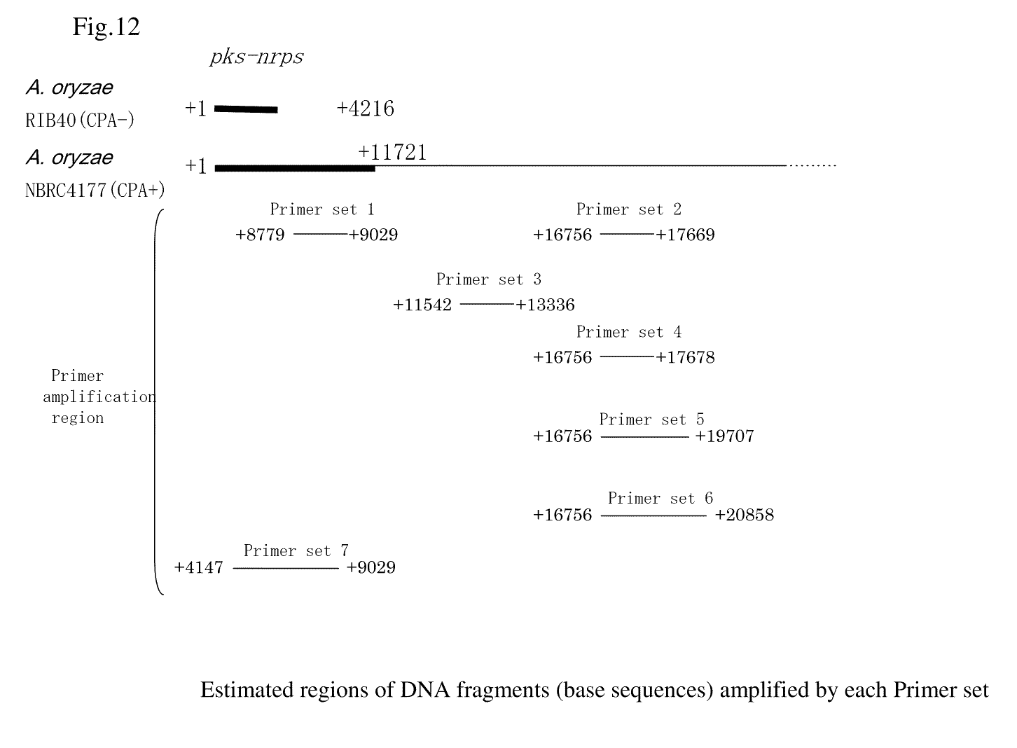

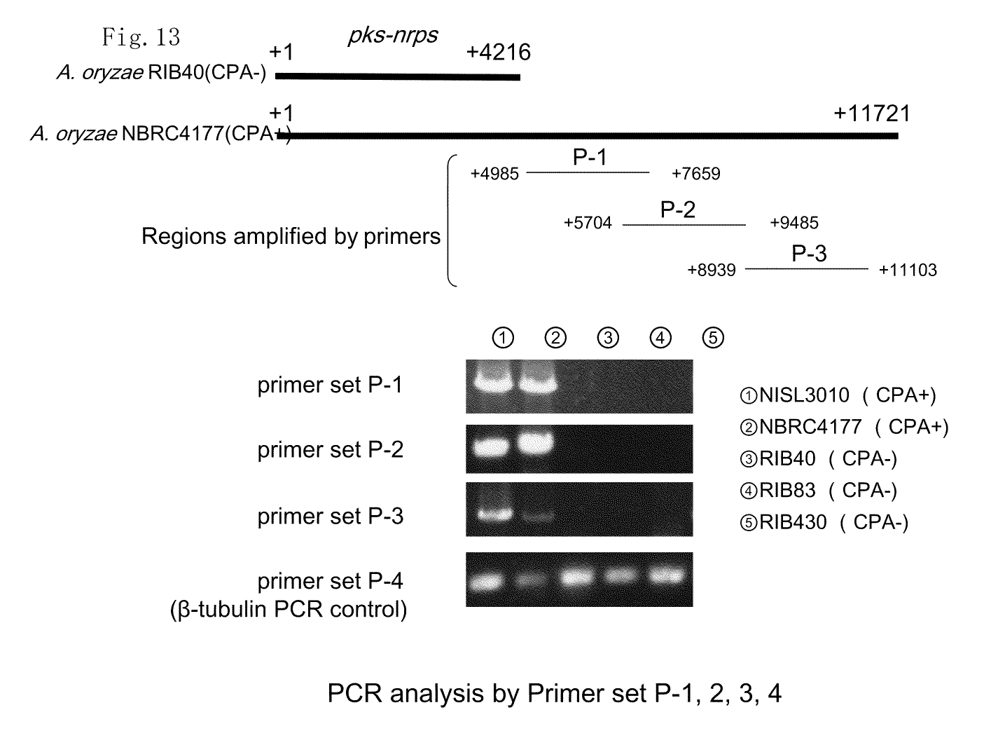

### BRIEF DESCRIPTION OF THE DRAWINGS

[FIG. 1] Photographs of electrophoresis illustrating a comparison of sequences by Southern analysis of an Aspergillus strain are included.

[FIG. 2] A photograph of electrophoresis illustrating the analysis results by a PCR of genomic DNA (third chromosome end) of an NBRC4177 strain is included.

[FIG. 3] A photograph of electrophoresis illustrating the presence or absence of a restriction enzyme fragment (approximately 2 kb) by Southern analysis of an Aspergillus strain is included.

[FIG. 4] A gene included in a cyclopiazonic acid biosynthesis gene cluster on the third chromosome of an Aspergillus strain and a positional relationship thereof are illustrated.

[FIG. 5] Photographs of electrophoresis illustrating the results of gene destruction included in a cyclopiazonic acid biosynthesis gene cluster by a PCR method are included. “Wt” indicates a wild strain and “TF1” and “TF2” indicate specific examples of each gene destruction strain.

[FIG. 6] Intermediary bodies of estimated cyclopiazonic acid synthesis pathways are illustrated.

[FIG. 7] A graph illustrating relative values of the amount of accumulation of the intermediates of the cyclopiazonic acid synthesis pathway of each gene destruction strain.

[FIG. 8] A photograph of electrophoresis illustrating the results of amplification of a DNA fragment by Primer set 1 is included.

[FIG. 9] A photograph of electrophoresis illustrating the results of amplification of DNA fragments by Primer sets 2 and 3 is included.

[FIG. 10] A photograph of electrophoresis illustrating the results of amplification of DNA fragments by Primer sets 4, 5, and 6 is included.

[FIG. 11] A photograph of electrophoresis illustrating the results of amplification of a DNA fragment by Primer set 7 is included.

[FIG. 12] Estimated regions of DNA fragments (base sequences) amplified by Primer sets 1 to 7 are illustrated.

[FIG. 13] Photographs of electrophoresis illustrating the results of amplification of DNA fragments by Primer sets P-1, 2, 3, and 4 are included.

### BEST MODE FOR CARRYING OUT THE INVENTION

As described in the examples of the present specification, the present invention first discovered that in a cyclopiazonic acid-producing strain of Aspergillus oryzae, there was a novel gene (polynucleotide) involved with cyclopiazonic acid production between a DACT-S gene involved with cyclopiazonic acid biosynthesis of the third chromosome and the telomere sequence and determined the base sequence by decoding the base sequence of a sequence amplified by an PCR primer designed with reference to a sequence of Aspergillus flavus and by decoding the base sequence of an amplification product of 3′-RACE with cDNA created by reverse transcription of an extracted RNA as a template. In addition, it was clarified that based on said base sequence, said gene was polyketide synthase-nonribosomal peptide synthetase (PKS-NRPS) having catalytic active domains of polyketide synthase (PKS) and nonribosomal peptide synthetase (NRPS). At the same time, it was found that in a strain that does not produce cyclopiazonic acid, 60% or more of the region on the 3′ side of a polynucleotide encoding PKS-NRPS was deleted due to the addition of a telomere repeat sequence from an intermediate point within the region.

[Polyketide Synthase-Nonribosomal Peptide Synthetase Gene and Protein]

Therefore, the novel polynucleotide found by the present invention encodes the following polypeptides.
(1) a polypeptide comprising the amino acid sequence depicted in SEQ ID NO: 2;
(2) a polypeptide comprising an amino acid sequence with one or several amino acids deleted, substituted or added in the amino acid sequence depicted in SEQ ID NO: 2 and having a polyketide synthase-nonribosomal peptide synthetase activity; or
(3) a polypeptide having 90% or more, preferably 95% or more, more preferably 98% or more homology (identity) as an overall average with the amino acid sequence depicted in SEQ ID NO: 2 and having a polyketide synthase-nonribosomal peptide synthetase activity.

Furthermore, it is generally known that polyketide synthase-nonribosomal synthetase is a multifunctional protein including a plurality of domains, it is estimated that the polyketide synthase-nonribosomal peptide synthetase comprising the amino acid sequence depicted in SEQ ID NO: 2 that was found by the inventors includes a plurality of functional domains as shown in Table 1 in the present specification, and it is desirable that the amino acids within the functional domains are retained in order to have a polyketide synthase-nonribosomal peptide synthetase activity. Therefore, as a specific example of the polypeptides shown in the above (2), it is preferred that among the amino acids depicted in SEQ ID NO: 2, the amino acids in a region other than the amino acids that constitute such a domain are deleted, substituted or added. In addition, as such an amino acid to be deleted, substituted or added, substitution of homologous amino acids (polar β nonpolar amino acid, hydrophobic β hydrophilic amino acid, positive β anionic amino acid, aromatic amino acid, etc.) is preferred.

In addition, a preferred example of said polynucleotide can include polynucleotides including the following polynucleotides.
(1) polynucleotides comprising the base sequence depicted in SEQ ID NO: 1;
(2) polynucleotides hybridizing under stringent conditions with polynucleotides comprising a base sequence complementary to the base sequence depicted in SEQ ID NO: 1 and encoding a polypeptide having a polyketide synthase-nonribosomal peptide synthetase activity.

Here, hybridization can be performed according to methods known in the art or methods that conform thereto such as the method described in Molecular cloning third ed. (Cold Spring Harbor Lab. Press, 2001), etc. Moreover, if a commercially available library is used, it can be performed according to the method described in the attached instruction.

Hybridization, for example, can be performed according to methods known in the art or methods that conform thereto such as the method described in Current protocols in molecular biology (edited by Frederick M. Ausubel et al., 1987), etc. Moreover, if a commercially available library is used, it can be performed according to the method described in the attached instruction.

In the present specification, “under a stringent condition” in hybridization between polynucleotides refers to a condition in which a so-called specific hybrid is formed and a non-specific hybrid is not formed. Therefore, “under a stringent condition” can specifically include, for example, a condition of a temperature of 60° C. to 68° C., a sodium concentration of 150 to 900 mM, preferably 600 to 900 mM, and pH6 to 8.

Such a polynucleotide that can hybridize with a polynucleotide comprising a base sequence complementary to a polynucleotide including the coding region depicted in SEQ ID NO: 1 can include, for example, a polynucleotide containing a base sequence in which the degree of homology with the entire base sequence of said DNA is 90% or more, preferably 95% or more, more preferably 98% or more as an overall average, etc.

The polynucleotide of the present invention can be acquired by colony hybridization or can also be prepared by amplification by a primer PCR using a strain belonging to genus Aspergillus or genus Penicillium that produces cyclopiazonic acid as a starting material and using an adequate probe that can be created based on sequence information described in the present specification, etc. or a primer PCR. It can be readily prepared by any method known to those skilled in the art. Examples of bacteria belonging to genus Aspergillus can include, for example, Aspergillus oryzae, Aspergillus flavus, and Aspergillus tamari, etc. Moreover, examples of bacteria belonging to genus Penicillium can include Penicillium camenberti, etc.

Furthermore, a PCR can be performed by accordingly selecting an adequate reaction condition known to those skilled in the art using, for example, a common thermal cycler such as 9600 manufactured by Perkin Elmer, etc. as a thermal cycler and a common commercialized product such as ExTaq DNA Polymerase (manufactured by Takara Shuzo Co, Ltd.) as thermally-resistant DNA polymerase.

Therefore, a polynucleotide prepared from such a strain is cDNA prepared by reverse transcription from genomic DNA or mRNA thereof. In addition, each gene of the present invention can also be prepared with chemical synthesis known to those skilled in the art.

Proteins of the present invention can include the following polypeptides.
(1) a polypeptide comprising the amino acid sequence depicted in SEQ ID NO: 2;
(2) a polypeptide comprising an amino acid sequence with one or several amino acids deleted, substituted or added in the amino acid sequence depicted in SEQ ID NO: 2 and having a polyketide synthase-nonribosomal peptide synthetase activity; or
(3) a polypeptide having 90% or more, preferably 95% or more, more preferably 98% or more homology (identity) as an overall average with the amino acid sequence depicted in SEQ ID NO: 2 and having a polyketide synthase-nonribosomal peptide synthetase activity.

Such a protein that is a variant of a polypeptide comprising the amino acid sequence depicted in SEQ ID NO: 2 can be readily created by accordingly combining methods known to those skilled in the art such as any method known to those skilled in the art, for example, site-specific mutagenesis, gene homologous recombination, primer extension, and PCR method.

Furthermore, identity between base sequences as well as amino acid sequences is determined by, for example, algorithms of Karlin and Altschul (Proc. Natl. Acad. Sci. USA 87:2264-2268, 1990 and Proc. Natl. Acad. Sci. USA 90:5873-5877, 1993). The BLAST program and the FASTA program using such algorithms are mainly used to search a sequence showing high sequence homology from a database for given sequences. These are available on, for example, the Internet website of U.S. National Center for Biotechnology Information.

[Creation of a Transformant that does not Produce Cyclopiazonic Acid]

As shown in the present specification, when said gene destruction strain (knockout strain) is further created by homologous recombination, cycloacetoacetyl L-tryptophan (CAT), which is an intermediate substance generated in the first stage of a cyclopiazonic acid biosynthesis pathway, was not synthesized and in consequence, it was confirmed that cyclopiazonic acid was also not produced.

Therefore, the present invention also relates to a method for producing by gene manipulation a transformant of microorganism belonging to genus Aspergillus or genus Penicillium that does not produce cyclopiazonic acid. A preferred specific example of such a transformant can include bacteria that do not express or produce polyketide synthase-nonribosomal peptide synthetase (PKS-NRPS).

Such a transformant that does not produce PKS-NRPS can be created by any gene manipulation means known to those skilled in the art.

For example, it is possible to cause PKS-NRPS not to be expressed by destroying a gene encoding PKS-NRPS (polynucleotide) using homologous recombination known to those skilled in the art, etc. However, as already described, Aspergillus bacteria have low homologous recombination frequency. Therefore, if a transformant of the present invention is created by such a method, it is preferred to use transformed bacteria in which a Ku gene such as Ku70 and Ku80 that is involved in non-homologous recombination is suppressed. Suppression of such a Ku gene can be performed by any method known to those skilled in the art. For example, it is possible to destroy a Ku gene using a Ku gene destruction vector by the method developed by the present inventors (Patent Document 4) or inactivate a Ku gene by an antisense RNA method using an antisense expression vector of the Ku gene. Transformed bacteria obtained in this manner have significantly increased homologous recombination frequency compared to the original bacteria before gene manipulation regarding such suppression of a Ku gene. Specifically, it is increased by at least 10 times, preferably at least approximately 60 times.

Examples of such transformed bacteria with increased homologous recombination frequency can include Aspergillus sojae ASKUPTR8 strains (wh, ΔpyrG, ku70::ptrA) and Aspergillus oryzae RkuN16ptr1 strains described in Patent Doc iment 4. Furthermore, Aspergillus sojae ASKUPTR8 strains were deposited in International Patent Organism Depositary, National Institute of Advanced Industrial Science and Technology located at Central 6, 1-1-1, Higashi, Tsukuba, Ibaraki, Japan on Dec. 2, 2004 and given Acceptance Number FERM P-20311. Subsequently, on Nov. 17, 2005, they were transferred to the international depository based on the Budapest Treaty on the International Recognition of the Deposit of Microorganisms for the Purposes of Patent Procedure and given Deposit (Accession) Number FERM BP-10453.

In addition, a Aspergillus oryzae A4177K strain, which is a Ku70 gene destruction strain, used in the examples of the present specification can be cited. This strain was deposited in International Patent Organism Depositary, National Institute of Technology and Evaluation located at 2-5-8, Kazusakamatari, Kisarazu-shi, Chiba, Japan on Oct. 16, 2007 and given Acceptance Number NITE AP-434.

[Method for Discriminating Cyclopiazonic Acid-Producing Ability]

As specifically shown in the present specification, in bacteria that do not produce cyclopiazonic acid such as Aspergillus oryzae, the sequence, depicted in SEQ ID NO: 1, of the 4,217th base and thereafter of a gene encoding polyketide synthetase-nonribosomal peptide synthetase (PKS-NRPS), which is an enzyme that catalyzes the first stage of cyclopiazonic acid biosynthesis, is deleted and the stop codon is not included. Instead, a telomere repeat sequence is added to the 3′ side of said base. A plurality of important active domains are present in PKS-NRPS and domains necessary for an activity are included in the region of 4,217th base and thereafter that has been deleted, and therefore it is estimated that an expression product of such a gene with the 3′ end side deleted cannot function as PKS-NRPS.

On the other hand, based on the analysis results in cyclopiazonic acid-producing bacteria, the region from the stop codon to the telomere of this gene is 17 to 18 kb, but in bacteria that do not produce cyclopiazonic acid, this region is also deleted. Moreover, because a PKS-NRPS gene is destroyed, cycloacetoacetyl L-tryptophan (CAT), which is an intermediate substance generated in the first stage in the cyclopiazonic acid biosynthesis pathway, is not synthesized and in consequence, cyclopiazonic acid is also not produced.

Furthermore, it is believed that bacteria belonging to genus Penicillium such as Penicillium camenberti have a group of genes (cluster) similar to Aspergillus oryzae related to cyclopiazonic acid production.

Based on the above, the presence of such a sequence specifically present only in cyclopiazonic acid-producing bacteria, i.e., a partial base sequence of a polynucleotide included in the 3′ region of a polynucleotide encoding polyketide synthase-nonribosomal peptide synthetase or the region from the stop codon to the telomere of said polynucleotide is useful as an index (target sequence) for discriminating cyclopiazonic acid-producing bacteria from non-producing bacteria.

Therefore, in a strain targeted for determination, it is possible to detect a partial base sequence of a polynucleotide included in the 3′ region of a polynucleotide encoding polyketide synthase-nonribosomal peptide synthetase of the present invention or the region from the stop codon to the telomere of said polynucleotide and to discriminate cyclopiazonic acid-producing ability in an Aspergillus strain or a Penicillium strain based on the presence or absence of said partial base sequence.

In particular, if a partial base sequence of a polynucleotide included in the region on the 3′ side of the polynucleotide encoding polyketide synthase-nonribosomal peptide synthetase is not detected, a polypeptide having a polyketide synthase-nonribosomal peptide synthetase activity is not expressed in said strain, and therefore it is possible to identify such a strain as a bacterium that does not produce cyclopiazonic acid.

Strains for the determination can include any Aspergillus strain such as Aspergillus oryzae and Aspergillus flavus as well as genus Penicillium such as Penicillium camenberti.

Here, one specific example of the “3′ region of a polynucleotide encoding polyketide synthase-nonribosomal peptide synthetase” can include a polynucleotide region comprising 4,217th to 11,721st base sequence in the base sequence depicted in SEQ ID NO: 1. Moreover, one example of a “region from the stop codon to the telomere of a polynucleotide encoding polyketide synthase-nonribosomal peptide synthetase” can include a sequence adjacent to the telomere in the 3′ downstream region of a PKS-NRPS gene of a cyclopiazonic acid-producing bacterium like the base sequence depicted in SEQ ID NO: 3.

The location and length of a partial base sequence for the detection are not particularly limited as long as they are included in the above region and can be detected by a method known to those skilled in the art, and one skilled in the art can select accordingly depending on the measuring method, etc. For example, if a partial base sequence is detected by DNA amplification by a PCR, said partial base sequence normally has 100 to 10,000 base pairs.

In the method of the present invention, a partial base sequence can be detected by any measuring method known to those skilled in the art. For example, it can be detected by a method known to those skilled in the art such as various PCR methods, etc. such as a RT-PCR method and a real-time PCR method using a primer or probe accordingly designed based on said base sequence, a Southern blot (analysis) method, an in situ hybridization method, and a microarray method (DNA chip).

A primer for amplification and a probe for hybridization that target the above base sequence, which is intended for detection, can be accordingly designed by one skilled in the art based on base information of each sequence described in the present specification or base sequence information available from public database, etc. These comprise adequate length depending on the application, for example, normally, about 10 to 100, preferably 20 to 40 continuous base sequences as for a primer for amplification. Moreover, a probe for hybridization normally comprises about 200 to 3,000, preferably 500 to 1,000 continuous base sequences.

Therefore, in the method for discriminating the cyclopiazonic acid-producing ability in the Aspergillus strain of the present invention, representative examples of partial base sequences useful as indexes (target sequences) for discriminating cyclopiazonic acid-producing bacteria from non-producing bacteria can include base sequences amplified by a PCR using Primer sets 1 to 7 and Primer sets P-1 to P-3 described in the examples of the present specification. Furthermore, as shown in FIG. 12, the partial base sequence amplified by Primer sets 1 to 7 have the whole or a part thereof included in the region from the 4,217th to the telomere in the base sequence of a polynucleotide encoding polyketide synthase-nonribosomal peptide synthetase depicted in SEQ ID NO: 1, or the partial base sequences amplified by Primer sets P-1 to P-3, as shown in FIG. 13, are included in the polynucleotide region comprising the 4,217th to 11,721st base sequences in the base sequence of a polynucleotide encoding polyketide synthase-nonribosomal peptide synthetase depicted in SEQ ID NO: 1.

The method of the present invention can be performed using an adequate measuring kit including the above primers or probes. Such primers or probes may be labeled with an adequately labeled substance such as any radioactive substance, fluorescent substance, and pigment known to those skilled in the art. Such a measuring kit can take an adequate configuration depending on the measuring principle to be utilized, etc. and additionally, other elements or components, for example, various reagents, enzymes, buffer solutions, and reaction plates (containers), etc. are included depending on the configuration and intended use, etc.

The present invention will be described in detail below based on examples, but the technical scope of the present invention is not limited by these descriptions. Furthermore, the means/conditions, etc. of each gene manipulation in the following examples can be performed according to standard gene engineering and molecular biological techniques known to those skilled in the art described in, for example, Japanese Published Unexamined Application No. Hei 8-80196, or Sambrook and Maniatis, in Molecular Cloning-A Laboratory Manual, Cold Spring Harbor Laboratory Press, New York, 1989; Ausubel, F. M. et al., Current Protocols in Molecular Biology, John Wiley & Sons, New York, N.Y., 1995, etc. unless otherwise stated. Moreover, the descriptions of the documents referenced in the present specification constitute the disclosure of the present specification and a part of the content.

### EXAMPLE 1

[Analysis of Cyclopiazonic Acid Productivity of the Test Strain]

In order to investigate whether a RIB40 strain as a standard strain of Aspergillus oryzae, in which genome information had been decoded was a cyclopiazonic acid-producing strain, with an Aspergillus oryzae NBRC4177 strain that was a known cyclopiazonic acid-producing strain as a positive control, the amount of cyclopiazonic acid accumulated in a CYA agar medium (3.0 g NaNO₃, 1.0 g K₂HPO₄, 0.5 g KCl, 0.5 g MgSO₄.7H₂O, 0.01 g FeSO₄. 7H₂O, 5.0 g Yeast Extract, 30.0 g Sucrose, 15.0 g Agar, 1000 ml Distilled water) was investigated after it was cultured for one week. The detection was performed using LC/MS (liquid chromatography/mass spectroscope) (1100 series high-performance liquid chromatography manufactured by Agilent, and QSTAR Elite mass spectroscope manufactured by Applied Viosystems). For the extraction, the culture medium and the fungus body were collected three times in spots of 6 mm in diameter and they were extracted with 800 μl of a solution in which ethyl acetate containing 1% formic acid, dichloromethane, and methanol were mixed at 3:2:1. Two centrifugal operations at 12,000 rpm for 5 minutes were performed in order to remove solids and a sample was obtained. For separation columns, ODS columns (Chemicals Evaluation and Research Institute L-column particle diameter 5 μm, inner diameter 2.1 mm, length 100 mm) were used, and for the eluent, 0.1 vol % formic acid water was A solution and 0.1 vol % formic acid in acetonitrile was B solution. 10 μl of the sample was separated by a method in which it was eluted with A solution 95% and B solution 5% from 0 minute to 5 minutes, followed by gradient of concentration so as to reach A solution 5% and B solution 95% by 25 minutes, and then to reach A solution 95% and B solution 5% in 35 minutes to 36 minutes. The flow rate of 0.2 ml per minute was used. For a mass analysis, electrospray ionization (ESI) was used as an ionization method with ion spray voltage of 5500 V, heater gas temperature of 450° C., nebulizer gas (GS1) of 50 psi, turbo gas (GS2) of 50 psi, curtain gas (Nitrogen) of 30 psi, and detection was performed in a positive ion detection mode.

In consequence, the peak of m/z=337.1 detected as a strong peak in the NBRC4177 strain, which was a cyclopiazonic acid-producing bacterium, was not detected in the RIB40 strain. Therefore, it was found that the RIB40 strain was a strain that did not produce cyclopiazonic acid.

[Preparation of Genomic DNA]

Aspergillus oryzae RIB40 strain, NBRC4177 strain, and NISL3010 strain were subjected to CYA liquid culture for 24 hours, and after the fungus body collected by filtration was frozen with liquid nitrogen, it was crushed using a mortar and a pestle. From the crushed fungus body, genomic DNA was extracted using a 50 mM tris-HCl buffer solution containing 2% SDS, 0.1M NaCl, and 10 mM EDTA, and after RNA was degraded with ribonuclease A, it was purified by phenol-chloroform treatment.

[Southern Analysis]

Rice molt fungus genome information (http://www.bio.nite.go.jp/dogan/MicroTop?GENOME_ID=ao) is genome sequencing information regarding an Aspergillus oryzae RIB40 strain, and with reference to this information, the base sequence on the upstream side of a DCAT-S gene was investigated and a region between a forward primer 5′-AGG GCT TGG TTA TGA AAA GTG TCC C-3′ (SEQ ID NO: 4) and a reverse primer 5′-CGC CTG ACG ATA CTG CAA ATG TC-3′ (SEQ ID NO: 5) was selected as a probe region. Moreover, as enzymes that cleave genomic DNA, BamHI and BglII (manufactured by Takara Bio Inc.) in which cleavage sequences are present on the centromere side from the primer region were selected in order to perform cleavage of the genome. For a Southern analysis, a method using digoxigenin (Roche Ltd.) was used and it was performed according to the method recommended by the manufacturer. In consequence, an analysis of the genome cleaved with BamHI and BglII showed that in the RIB40 strain, because the telomere existed, only bands of 3.3 kb and 2.7 kb were obtained, but in the NBRC4177 strain and the NISL3010 strain, bands of approximately 15 kb and 5 kb were obtained (FIG. 1). Therefore, it was believed that in these two strains, the telomeres were added in the region more downstream compared to RIB40. Therefore, with reference to genome information of Aspergillus flavus, which is known as a related species of Aspergillus oryzae, a PCR was performed with genomic DNA of the NBRC4177 strain as a template using a reverse primer 13452:5′-GCACAAACCGTGAAATGATCCTTTTCACTG-3′ (SEQ ID NO: 7), a reverse primer 14433:5′-CTCCACGAGTGCGGGGAGTGGGCCAATAG-3′ (SEQ ID NO: 8), a reverse primer 15463:5′-GGCCGCACGTGACTTCAGTCATGTGATC-3′ (SEQ ID NO: 9), a reverse primer 16612:5′-CGGATTGTCCCACGCTCATAGTGTTTTGC-3′ (SEQ ID NO: 10), and a reverse primer 17850:5′-GTCGACCGTTTCCTGTCTTTACCACAC-3′ (SEQ ID NO: 11) relative to a forward primer 12539:5′-GGGAGGAACAATTGTATTGCTGGCTTTG-3′ (SEQ ID NO: 6) as PCR primers in which this downstream region is the target sequence, and in consequence, amplification of DNA was observed in the reverse primers except for the reverse primer 17850 (FIG. 2). Moreover, genomic DNA of the RIB40 strain, the NISL3010 strain, and the NBRC4177 strain were cleaved with SalI and BglII and a Southern analysis using fragments of the region amplified by a forward primer 5′-CGGGTCTGCAGGCACGCATAAAGACTG-3′ (SEQ ID NO: 12) and a reverse primer 5′-ATCGCATGTGCTGTATGGATCCGACTATCC-3′ (SEQ ID NO: 13) as probes, and in consequence, fragments of approximately 2 kb were detected only in the NISL3010 strain and the NBRC4177 strain in the both restriction enzymes (FIG. 3). Therefore, a telomere addition site was predicted with reference to these results and an attempt to clone the telomere addition region by an inverse PCR was made. In consequence, it was clarified that in the NBRC4177 strain, the telomere was added in approximately 17 to 18 kb more downstream compared to the RIB40 strain. The base sequence around the telomere addition site was as depicted in SEQ ID NO: 2.

Moreover, a Northern analysis indicated that a PKS gene in which the telomere has been added in the RIB40 strain has been expressed in the NBRC4177 strain and a transcription product thereof was approximately 10 kb. Therefore, based on the results that have been obtained, the coding region was predicted to be 11.7 kb and a 3′-RACE analysis was performed. Generacer kit (Invitrogen) was used for reverse transcription and a primer 5′-CAGATCAAGGTCGGGATTTCAGC-3′ (SEQ ID NO: 14) and a primer provided in the kit were used for a PCR with cDNA as a template. The resulting product was cloned with a TA-cloning kit (Invitrogen) in order to decode the base sequence. Based on this sequence, a PCR primer was additionally synthesized and determination of the base sequence was repeated in order to decode the whole-length base sequence depicted in SEQ ID NO: 1. When the amino acid sequence predicted from the base sequence of this gene was analyzed by a Pfam program (http://www.sanger.ac.uk/Software/Pfam/), it was clarified that it encoded a PKS-NRPS hybrid-type enzyme having catalytic domains of PKS and NRPS. Furthermore, each domain and a position thereof (numbers of start and end of amino acids and bases) clarified by the above analysis of the amino acid sequence with the Pfam program is shown below in Table 1.

TABLE 1

| Domain | Amino acid Start | End | Base start | End |
|---|---|---|---|---|
| ketoacyl-synt | 3 | 254 | 9 | 762 |
| Ketoacyl-synt_C | 262 | 385 | 786 | 1155 |
| Acyl_transf_1 | 555 | 887 | 1665 | 2661 |
| adh_short | 2064 | 2228 | 6192 | 6684 |
| KR | 2064 | 2241 | 6192 | 6723 |
| Condensation | 2501 | 2793 | 7503 | 8379 |
| AMP-binding | 2980 | 3392 | 8940 | 10176 |
| PP-binding | 3481 | 3545 | 10443 | 10635 |
| NAD_binding_4 | 3594 | 3815 | 10782 | 11445 |
| Thiolase_N | 168 | 203 | 504 | 609 |
| PP-binding | 2360 | 2425 | 7080 | 7275 |
| Epimerase | 3592 | 3668 | 10776 | 11004 |

### EXAMPLE 2

[Creation of a Transformant of an Aspergillus Strain that does not Produce Cyclopiazonic Acid]

As genes included in a cyclopiazonic acid biosynthesis gene cluster on the third chromosome, cpaC (AO090026000003) and cpaD (AO090026000004), which are other genes positioned around the DCAT-S gene (cpaB: AO090026000002), are predicted (FIG. 4). Therefore, systematic destruction was performed on the PKS-NRPS gene (cpaA: AO090026000001) identified in the present invention and those genes.

Prior to each gene destruction, in order to increase the gene destruction efficiency in a cyclopiazonic acid-producing strain, an A4177K strain, which is a strain in which the gene of protein Ku70 involved with non-homologous recombination in the Aspergillus oryzae NBRC4177 strain is destroyed, was created according to the method described in the above Patent Document 4 and the previous report of the inventors (Takahashi et al., Mol. Gen. Genet. (2006) 275: 461-470) and furthermore to use a PyrG gene encoding orotidine 5-phosphate carboxylase as a selective marker for transformation, using a DNA fragment sequence in which a part of a PyrG gene is deleted, transformation was performed with a means known to those skilled in the art using a protoplast PEG method with 5′-phosphoorotidine acid resistance as an index, in order to obtain a A4177KP strain, which was a strain that retained a partially deleted PyrG sequence. For the DNA fragments used for destruction of these Ku70 and PyrG genes, DNA fragments amplified by a PCR from the genome sequence of the abovementioned Aspergillus oryzae RkuN16ptr1 strain were used. Moreover, the DNA fragment for gene destruction was performed according to the report of Tamano, et al. (Tamano et al., Biosci. Biotechnol. Biochem., (2007) 71: 926-934).

The method for preparing the gene destruction fragments of the cpaA gene will be described in detail below. With the genome of the Aspergillus oryzae NBRC4177 strain as a template, using LU and LL of Primer cpaA, a fragment including the sequence on the upstream side of the cpaA gene was amplified by a PCR. Similarly, a fragment including the sequence on the downstream side of the cpaA gene was amplified using RU and RL of Primer cpaA. Furthermore, a fragment of the selective marker pyrG gene was amplified by PU and PL of Primer cpaA. Because PU and PL of Primer cpaA have a sequence complementary to the sequence fragment on the upstream side and the sequence fragment on the downstream side of the cpaA gene that has been amplified above, one fragment in which the three fragments have been fused was obtained by performing a PCR reaction with these three fragments as mutual primers. Because this fragment has a structure in which the upstream and downstream partial sequences of the cpaA gene were present at the both ends of the pyrG gene, it becomes a gene destruction fragment that performs substitution destruction. With a similar procedure, a destruction fragment of each gene included in the above cyclopiazonic acid biosynthesis gene cluster was created using the following primer sequences.

Primer cpaA
LU:
(SEQ ID NO: 15)
5′-TGCTCGCCGTTAGCCTTTCGTTTCAC-3′
LL:
(SEQ ID NO: 16)
5′-AGCGGCAGAACTGGCAGCAGATAATAGAG-3′
PU:
(SEQ ID NO: 17)
5′-TGCTGCCAGTTCTGCCGCTACAACAGACGTACCCTGTGATGTTC-3′
PL:
(SEQ ID NO: 18)
5′-TTAACACGCTTTCGTCGCTAACTGCACCTCAGAAGAAAAGGATG-3′
RU:
(SEQ ID NO: 19)
5′-AGCGACGAAAGCGTGTTAACCAAGGTATG-3′
RL:
(SEQ ID NO: 20)
5′-TTTGAGCGCAATCGGGATGAGTAATGTAG-3′
Primer cpaB
LU:
(SEQ ID NO: 21)
5′CTGCCAAAGCCCTTCTACGTGCTGAGTC-3′
LL:
(SEQ ID NO: 22)
5′-GTACGTCTGTTGT GGCAGCCTTGATTGCGTCAAACATGAG-3′
PU:
(SEQ ID NO: 23)
5′-TGACGCAATCAAGGCTGCCACAACAGACGTACCCTGTGATGTTC-3′
PL:
(SEQ ID NO: 24)
5′GGATTGCCAGTGGAGTGGCAACTGCACCTCAGAAGAAAAGGATG-3′
RU:
(SEQ ID NO: 25)
5′-CTGAGGTGCAGTT GCCACTCCACTGGCAATCCTCGAGGAG-3′
RL:
(SEQ ID NO: 26)
5′-GCAGCAGCACTGAACGCTTCGAAGGTATG-3′
Primer cpaC
LU:
(SEQ ID NO: 27)
5′-TCTTTCCACCGTCGCCTATCTTGCTTTG-3′
LL:
(SEQ ID NO: 28)
5′-GTACGTCTGTTGTTTCCAGGACATCGCCAGATGTGTGAG-3′
PU:
(SEQ ID NO: 29)
5′-ATCTGGCGATGTCCTGGAAACAACAGACGTACCCTGTGATGTTC-3′
PL:
(SEQ ID NO: 30)
5′-CCCTCATTCAAGGCAGCGGAACTGCACCTCAGAAGAAAAGGATG-3′
RU:
(SEQ ID NO: 31)
5′-CTGAGGTGCAGTTCCGCTGCCTTGAATGAGGGCTACGTC-3′
RL:
(SEQ ID NO: 32)
5′-CCCCCACAGCAAGGTCGAGTAATCTGAC-3′
Primer cpaD
LU:
(SEQ ID NO: 33)
5′-CGGTTGCTTGCGAAGGGATTTTCAGATG-3′
LL:
(SEQ ID NO: 34)
5′-GTACGTCTGTTGTTGGCGCTAAGAGCTGTTGCTGTCGTCTC-3′
PU:
(SEQ ID NO: 35)
5′-GCAACAGCTCTTAGCGCCAACAACAGACGTACCCTGTGATGTTC-3′
PL:
(SEQ ID NO: 36)
5′-GCGCTTGGCATTTTCGTTCAACTGCACCTCAGAAGAAAAGGATG-3′
RU:
(SEQ ID NO: 37)
5′-CTGAGGTGCAGTTGAACGAAAATGCCAAGCGCAAAGTCATC-3′
RL:
(SEQ ID NO: 38)
5′-CTCTGATCCAGGGGCTAGCTCCCAATC-3′

Using gene destruction fragments obtained in this manner, transformation was performed with the Aspergillus oryzae 4177K strain as a host and a gene destruction strain was obtained. Confirmation of the gene destruction was performed by a PCR using a primer that started amplifying at an outer sequence from the gene destruction fragment used, with the change in amplification fragment length equivalent to the gene of the selective marker as an index. Frame formats regarding the confirmation of the gene destruction by a PCR and the results of the confirmation regarding each gene destruction strain are shown in FIG. 5. The sequences of the primers used for the confirmation are as follows.

Primer cpaA
CU:

| | |
|---|---|
| 5′-CGGCGAGATAGTGGCTGCCTATGCTC-3′ | (SEQ ID NO: 39) |
| CL: | |
| 5′CAGGGTCAAGCCCCAGAACATTCATG-3′ | (SEQ ID NO: 40) |
| Primer cpaB | |
| CU: | |
| 5′-GGCACCCGAAAGCTGAGCAATGGAG-3′ | (SEQ ID NO: 41) |
| CL: | |
| 5′-TGGCGCGTGGCAACAAGGTCTATG-3′ | (SEQ ID NO: 42) |
| Primer cpaC | |
| CU: | |
| 5′-AGGCCCGAGATGAGCAATCTTGGGAATC-3′ | (SEQ ID NO: 43) |
| CL: | |
| 5′-ACCGCGTTTGTGCGAGACCGTACTTGAC-3′ | (SEQ ID NO: 44) |
| Primer cpaD | |
| CU: | |
| 5′-GCGTCTCTGGCATTCGTACCATCTATG-3′ | (SEQ ID NO: 45) |
| CL: | |
| 5′-ATACTGGAGACACAGCGCACACGATAC-3′ | (SEQ ID NO: 46) |

In addition, an analysis of a metabolite of each of the obtained gene destruction strains was performed by LC/MS. Intermediary bodies of the cyclopiazonic acid synthesis pathway estimated in Penicillium camenberti are cycloacetoacetyl-L-tryptophan, beta-cyclopiazonic acid, and alpha-cyclopiazonic acid (FIG. 6) and the exact molecular weight of each of them is 270.1004, 338.1630, and 336.1474. In the LC/MS analysis, the peak of a value in which 1.0078 was added to each molecular weight was investigated in order to detect protonated molecules, and the graph in FIG. 7 shows the relative values of the amount of accumulation in each gene destruction strain. It is clear from the graph that in the strain (DcpaA) in which the PKS-NRPS gene was destroyed, no cyclopiazonic acid biosynthesis intermediary bodies were accumulated. In contrast, it was confirmed that in the strains in which other genes were destroyed, cyclopiazonic acid biosynthesis intermediary bodies were accumulated. Therefore, it was found that a safer strain in which no intermediary bodies of cyclopiazonic acid were produced could be obtained by destroying the PKS-NRPS gene.

### EXAMPLE 3

[Method for Discriminating Cyclopiazonic Acid-Producing Ability]

Based on the results of Example 1, it was found that in Aspergillus oryzae, the base sequence of the third chromosome end region was different between cyclopiazonic acid-producing bacteria and non-producing bacteria, cyclopiazonic acid-producing bacteria retained a full-length PKS-NRPS gene, which is deleted in non-producing bacteria, and a sequence of approximately 17 to 18 kb was present by the telomere. Therefore, a PCR primer in which the region that only such cyclopiazonic acid-producing bacteria have is the amplification target sequence was created based on the genome information of Aspergillus flavus in the similar manner to Example 1 and an amplification test by a PCR was performed on a plurality of Aspergillus oryzae strains (a NISL3010 strain and a NBRC4177 strain as cyclopiazonic acid-producing bacteria, and a RIB40 strain as a bacterium that does not produce cyclopiazonic acid).

Firstly, an Aspergillus oryzae strain was subjected to liquid culture for 24 hours in a CYA culture medium, and after the fungus body collected by filtration was frozen with liquid nitrogen, it was crushed using a mortar and a pestle. From the crushed fungus body, genomic DNA was extracted using a 50 mM tris-HCl buffer solution containing 2% SDS, 0.1M NaCl, and 10 mM EDTA, and after RNA was degraded with ribonuclease A, it was purified by phenol-chloroform treatment. Genomic DNA was extracted from each strain and a PCR analysis was performed with those as templates. Furthermore, for a reaction solution, genomic DNA 1 μl, primer set 2 μl, Ex taq buffer 5 μl, 25 mM dNTP 5 μl, Ex-Taq 0.8 μl, and distilled sterile water 36.2 μl were used. For a PCR reaction, using PTC220 manufactured by Bio-Rad, 30 cycles of 94° C. for 2 minutes, 94° C. for 20 seconds, 60° C. for 20 seconds, and 72° C. for 20 seconds were repeated. After the reaction product was electrophoresed with 0.8% agarose gel, etc., the amplification product was visualized as a band under ultraviolet exposure by staining with ethidium bromide. Furthermore, the PCR primer sequences used are as follows.

Primer set 1
Forward primer:
(SEQ ID NO: 47)
5′-GTCGCCACTTCTGCTCCCATCAAC-3′
Reverse primer:
(SEQ ID NO: 48)
5′-GGGCACAAGACTGCGATTGTGTCTC-3′
Primer set 2
Forward primer:
(SEQ ID NO: 49)
5′-GGGAGGAACAATTGTATTGCTGGCTTTG-3′
Reverse primer:
(SEQ ID NO: 50)
5′-GCACAAACCGTGAAATGATCCTTTTCACTG-3′
Primer set 3
Forward primer:
(SEQ ID NO: 51)
5′-CCGGTGAAGAGCTTCAAGGAATATATG-3′
Reverse primer:
(SEQ ID NO: 52)
5′-CAGTACGCAAATCGGAATCAAGTTGCAGAG-3′
Primer set 4
Forward primer:
(SEQ ID NO: 53)
5′-GGGAGGAACAATTGTATTGCTGGCTTTG-3′
Reverse primer:
(SEQ ID NO: 54)
5′-CTCCACGAGTGCGGGGAGTGGGCCAATAG-3′
Primer set 5
Forward primer:
(SEQ ID NO: 55)
5′-GGGAGGAACAATTGTATTGCTGGCTTTG-3′
Reverse primer:
(SEQ ID NO: 56)
5′-GGCCGCACGTGACTTCAGTCATGTGATC-3′
Primer set 6
Forward primer:
(SEQ ID NO: 57)
5′-GGGAGGAACAATTGTATTGCTGGCTTTG-3′
Reverse primer:
(SEQ ID NO: 58)
5′-CGGATTGTCCCACGCTCATAGTGTTTTGC-3′
Primer set 7
Forward primer:
(SEQ ID NO: 59)
5′-ACTCATGATGCGGCGATGTTCTCTCA-3′
Reverse primer:
(SEQ ID NO: 60)
5′-GGGCACAAGACTGCGATTGTGTCTC-3′

The DNA fragments amplified by each of the above primer sets were separated with 0.8% agarose gel, and after staining with ethidium bromide, they were visualized as a band by ultraviolet exposure. The results are shown in FIGS. 8 to 11. Based on these results, it was confirmed that in the NISL3010 strain and the NBRC4177 strain, which are cyclopiazonic acid-producing bacteria, the base sequences were amplified by each of the above primer sets, but in the RIB40 strain, which is a bacterium that does not produce cyclopiazonic acid, the base sequence was not amplified at all. Moreover, the estimated regions of the base sequences amplified by each of the above primer sets are shown in FIG. 12. However, because they are based on the sequence information of Aspergillus flavus, the positions of the amplification regions in Aspergillus oryzae may be displaced by about 0 to 1,000 bases. As shown in FIG. 12, it is clear that the partial base sequence amplified by Primer sets 1 to 7 have the whole or a part thereof included in the region from the 4,217th to the telomere in the base sequence of a polynucleotide encoding polyketide synthase-nonribosomal peptide synthetase depicted in SEQ ID NO: 1.

In addition, a similar experiment was performed using a NISL3010 strain and a NBRC4177 strain as cyclopiazonic acid-producing bacteria of an Aspergillus oryzae strain, a RIB40 strain as a bacterium that does not produce cyclopiazonic acid, and furthermore a RIB83 strain and a RIB430 strain that had been confirmed to be bacteria that do not produce cyclopiazonic acid by the method described in Example 1. For a reaction solution, genomic DNA 1 μl (200 ng), primer set 2 μl, Ex taq buffer 5 μl, 25 mM dNTP 5 μl, Ex-Taq 0.2 μl, and distilled sterile water 36.5 μl were used. For a PCR reaction, using PTC220 manufactured by Bio-Rad, 30 cycles of 94° C. for 2 minutes, 94° C. for 20 seconds, 60° C. for 20 seconds, and 72° C. for 4 minutes were repeated. After the reaction product was electrophoresed with 0.8% agarose gel, etc., the amplification product was visualized as a band under ultraviolet exposure by staining with ethidium bromide. Furthermore, the PCR primer sequences used are as follows. The results are shown in FIG. 13. Based on these results, it was confirmed that in the NISL3010 strain and the NBRC4177 strain, which are cyclopiazonic acid-producing bacteria, the base sequences were amplified by each of the above primer sets, but in the RIB40 strain, RIB83 strain and RIB430 strain, which are bacteria that do not produce cyclopiazonic acid, the base sequence was not amplified at all. Moreover, as shown in FIG. 13, it is clear that the partial base sequence amplified by Primer sets P-1 to P-3 are included in the polynucleotide region comprising the 4,217th to the 11,721st base sequences in the base sequence of a polynucleotide encoding polyketide synthase-nonribosomal peptide synthetase depicted in SEQ ID NO: 1.

Primer set P-1
+4985 Forward primer:
(SEQ ID NO: 61)
5′-TTTCCCTGGTGGAGCTTGACGAGCC-3′
+7659 Reverse primer:
(SEQ ID NO: 62)
5′-CTCATGCATAGAGACAGCGTGTTCC-3′
Primer set P-2
+5704 Forward primer:
(SEQ ID NO: 63)
5′-GAGCCCGATGAGTTACTTGCTGCTG-3′
+9485 Reverse primer:
(SEQ ID NO: 64)
5′-CCTCCGTTCATGATGGCATTGAGAGTCTG-3′
Primer set P-3
+8939 Forward primer:
(SEQ ID NO: 65)
5′-GCTATTTGCAGCTCCAAGCGCAAAG-3′
+11103 Reverse primer:
(SEQ ID NO: 66)
5′-TTGCGACTGGAGGGCAAATTCGGCG-3′
Primer set P-4

(Control primer: Refer to Tominaga et al., Appl. Environ. Microbiol. (2006) 72: 484-490.)

| Control forward primer: | |
|---|---|
| 5′CCAAGAACATGATGGCTGCT-3′ | (SEQ ID NO: 67) |
| Control reverse primer: | |
| 5′-CTTGAAGAGCTCCTGGATGG-3′ | (SEQ ID NO: 68) |

Based on the above results, the bands detected in cyclopiazonic acid-producing bacteria using each of the above PCR primer sets were not detected in bacteria that did not produce cyclopiazonic acid, and it was indicated that it was possible to discriminate the cyclopiazonic acid-producing ability in the Aspergillus strain by the method of the present invention, thereby cyclopiazonic acid-producing bacteria can be discriminated from non-producing bacteria.

### INDUSTRIAL APPLICABILITY OF THE INVENTION

It has become possible to create a transformant (strain) of Aspergillus bacteria, etc. that does not express polyketide synthase-nonribosomal peptide synthetase (PKS-NRPS) using the present invention. In consequence, it has become possible to industrially utilize safe Aspergillus bacteria, etc. that cannot produce cyclopiazonic acid, which is one type of mycotoxin, and cycloacetoacetyl L-tryptophan (CAT), which is a precursor thereof.

1. An isolated polynucleotide encoding (1) a polypeptide comprising the amino acid sequence depicted in SEQ ID NO: 2; (2) a polypeptide comprising an amino acid sequence with one or several amino acids deleted, substituted or added in the amino acid sequence depicted in SEQ ID NO: 2 and having a polyketide synthase-nonribosomal peptide synthetase activity; or (3) a polypeptide having 90% or more homology (identity) as an overall average with the amino acid sequence depicted in SEQ ID NO: 2 and having a polyketide synthase-nonribosomal peptide synthetase activity.

2. An isolated polynucleotide comprising: (1) a base sequence depicted in SEQ ID NO: 1; or (2) a polynucleotide capable of hybridizing under stringent conditions with polynucleotides comprising a base sequence complementary to the base sequence depicted in SEQ ID NO: 1 and encoding a polypeptide having a polyketide synthase-nonribosomal peptide synthetase activity.

3. An isolated polynucleotide according to claim 1 or 2 that has a base sequence found in the genome of Aspergillus oryzae.

4. An isolated polynucleotide according to claim 1 or 2 that is cDNA.

5. An isolated polyketide synthase-nonribosomal peptide synthetase comprising: (1) a polypeptide comprising the amino acid sequence depicted in SEQ ID NO: 2; (2) a polypeptide comprising an amino acid sequence with one or several of the amino acids deleted, substituted or added in the amino acid sequence depicted in SEQ ID NO: 2 and having a polyketide synthase-nonribosomal peptide synthetase activity; or (3) a polypeptide having 90% or more homology (identity) as an overall average with the amino acid depicted in SEQ ID NO: 2 and having a polyketide synthase-nonribosomal peptide synthetase activity.

6. A method for producing by gene manipulation a transformant of a microorganism belonging to genus Aspergillus or genus Penicillium that does not produce cyclopiazonic acid.

7. The method according to claim 6, wherein the transformant that does not produce cyclopiazonic acid is a bacterium that does not produce cycloacetoacetyl L-tryptophan.

8. The method according to claim 6, wherein the transformant that does not produce cyclopiazonic acid is a bacterium that does not express polyketide synthase-nonribosomal peptide synthetase.

9. The method according to claim 6, wherein the gene manipulation destroys a polynucleotide encoding a polyketide synthase-nonribosomal peptide synthetase comprising (1) an amino acid sequence depicted in SEQ ID NO: 2; (2) an amino acid sequence with one or several of the amino acids deleted, substituted or added in the amino acid sequence depicted in SEQ ID NO: 2; or (3) an amino acid sequence sharing 90% or more identity with the amino acid depicted in SEQ ID NO: 2.

10. The method according to claim 9, wherein the gene manipulation is performed on a strain in which a Ku gene involved in non-homologous recombination is destroyed.

11. The method according to claim 6, wherein the microorganism is an Aspergillus oryzae strain.

12. The method according to claim 11, wherein an Aspergillus oryzae strain in which the homologous recombination frequency has been elevated is used.

13. The method according to claim 12, wherein a transformed bacterium in which the homologous recombination frequency has been elevated is an Aspergillus oryzae A4177K strain.

14. A transformant that does not produce cyclopiazonic acid, obtained by the method of manufacturing according to claim 6.

15. The transformant that does not produce cyclopiazonic acid according to claim 14 that is an Aspergillus oryzae strain.

16. A method for discriminating cyclopiazonic acid-producing ability, wherein a partial base sequence of a polynucleotide included in the 3′ region of the polynucleotide encoding the polyketide synthase-nonribosomal peptide synthetase according to claim 1 or 2 or in the region from the stop codon to the telomere of said polynucleotide is detected, and cyclopiazonic acid-producing ability in an Aspergillus strain or a Penicillium strain is discriminated based on the presence or absence of said partial base sequence.

17. A method for identifying a strain that does not produce cyclopiazonic acid, wherein a partial base sequence of a polynucleotide included in the region on the 3′ side of the polynucleotide encoding the polyketide synthase-nonribosomal peptide synthetase according to claim 1 or 2 is detected, and a bacterium that does not produce cyclopiazonic acid in an Aspergillus strain or a Penicillium strain is identified based on the presence or absence of said partial base sequence.

18. The method according to claim 16, wherein the Aspergillus strain is Aspergillus oryzae.

19. The method according to claim 16, wherein the 3′ region of the polynucleotide encoding the polyketide synthase-nonribosomal peptide synthetase is the 4,217th to the 11,721st in the base sequence depicted in SEQ ID NO: 1.

20. The method according to claim 16, wherein the polynucleotide region included between the polynucleotide encoding the polyketide synthase-nonribosomal peptide synthetase and the telomere sequence present in its 3′ downstream region has the base sequence depicted in SEQ ID NO: 3.

21. The method according to claim 16, wherein the presence or absence of the partial base sequence of the polynucleotide is detected by a polymerase chain reaction.

22. The method according to claim 16, wherein the presence or absence of the partial base sequence of the polynucleotide is detected by a Southern analysis.
